(19) United States
(12) Patent Application Publication
(10) Pub. No.: US 2011/0008438 A1
(43) Pub. Date: Jan. 13, 2011

(54) Bone Repair Composition and a Method of Making the Same

(76) Inventors: Peter Myint, Oswestry (GB); Jan Herman Kuiper, Shropshire (GB)

Correspondence Address: LICATA & TYRRELL P.C., 66 E. MAIN STREET, MARLTON, NJ, 08053, US

(21) Appl. No.: 12/812,100

(22) PCT Filed: Jan. 9, 2009
(86) PCT No.: PCT/GB09/50017

§ 371 Date: Sep. 29, 2010

(30) Foreign Application Priority Data
Jan. 9, 2008 (GB) 0800370.9
Jan. 9, 2008 (GB) 0800371.7

(51) Int. Cl. A61K 9/00; A61K 38/18; A61P 43/00

(52) U.S. Cl. 424/484; 514/7.6; 977/908

(57) ABSTRACT

Bone repair composition and method of making the same, the bone repair composition being formed by firstly mixing a first aqueous calcium phosphate suspension with bone graft granules to form an intermediate mixture. The intermediate mixture is then mixed with a second aqueous calcium phosphate suspension, wherein the first aqueous calcium phosphate suspension contains a lower weight concentration of calcium phosphate than the second aqueous calcium phosphate suspension.

The present invention concerns a bone repair composition and a method of making the same and, in particular, a bone repair composition for use in impaction grafting, for example in revision total joint replacement surgery.

In this connection, total joint replacement surgery, in particular for the hip or knee, is relatively successful. Nevertheless, over time, joint prosthesis failure can occur, necessitating revision surgery. The most common reason for prosthesis failure is aseptic loosening, where, for various reasons, bone around the prosthesis is progressively resorbed until the prosthesis has lost its fixation.

During joint replacement surgery, coping with the bone loss caused by aseptic loosening is extremely challenging. Some revision techniques involve using larger prostheses and/or more bone cement to fill the spaces left by resorbed bone. However, such techniques do not attempt to counteract the loss of bone tissue. As such, yet further revision surgeries will result in a vicious cycle of ever reducing bone mass and, consequently, failure rates are much higher than for initial replacement operations. This is a particular problem in revision hip replacement surgery, as the femoral stem of the prosthesis must be inserted into the medullary canal of the femur and be supported by the surrounding bone mass.

To overcome the above issue, techniques have been developed to try to replace lost bone before implanting a new replacement joint prostheses. Impaction bone grafting using morselized bone is one such method that has been used for revision hip replacement surgery. In this method, morselized allograft bone granules, typically 1-5 mm in diameter, are packed into the medullary canal. A cannulated tamp is positioned during the packing process and, once removed, forms a neo-medullary canal. A revision femoral stem prosthesis is then implanted into the neo-medullary canal using PolyMethylMethAcrylate bone cement. Tight packing of the bone chips promotes stability of the revision prosthesis, and spaces between bone chips allow ingrowth of blood vessels and invasion by bone cells, promoting replacement of the bone graft by new viable bone. These spaces also allow penetration of PMMA bone cement. There is therefore a balance between the mechanical demands of enabling initial stability of the prosthesis and achieving a consistency suitable for long term enhancement of bone development.

Whist the above technique has been successful, it involves the use of morselized allograft bone. This is often prepared in the operating theatre by grinding femoral heads. However, the continued use of allograft bone is of increasing concern because of high costs, limited supply and the risk of disease transmission.

To address these issues, morselized allograft bone has been mixed with synthetic bone substitutes, such as calcium phosphate granules, sized to match the morselized allograft bone, in order to reduce the amount of allograft used. However, the synthetic bone substitutes have different mechanical and handling properties compared to allograft bone, so, though they can mitigate the problems of expense, supply and disease transmission, surgeons are often reluctant to use them in practice. Furthermore, recommended practice is to mix bone substitutes with allograft bone, usually in a 50:50 ratio, and therefore the problems with the allograft material are not wholly avoided.

A particular problem with known bone substitutes is that during the impaction procedure outlined above, a large number of the synthetic bone substitute particles are displaced and fall down the narrow neo-medullary canal each time the cannulated tamp is withdrawn. This perturbs or unsettles the neo-medullary canal and compromises its interface with the prosthesis femoral stem. It has been suggested that this is because the synthetic bone substitute particles are less “sticky” or “cohesive” than allograft bone. To improve the cohesiveness of the materials, some surgeons add clotted blood to the mixture of morselized allograft bone and synthetic bone substitute. Although this offers some improvement, it still fails to produce a mixture as cohesive as pure morselized allograft bone, which remains the preferred material for this procedure.

Accordingly, the present invention seeks to overcome the above problems associated with the prior art.

According to an aspect of the present invention there is provided a bone repair composition formed by firstly mixing a first aqueous calcium phosphate suspension with bone graft granules to form an intermediate mixture, and secondly mixing the intermediate mixture with a second aqueous calcium phosphate suspension, wherein said first aqueous calcium phosphate suspension contains a lower weight concentration of calcium phosphate than the second aqueous calcium phosphate suspension.

In this way, the calcium phosphate suspension forms a paste like binder for the bone graft granules, thereby enhancing cohesion between the bone graft granules. In particular, during the mixing process, the first, lower concentration, aqueous calcium phosphate suspension coats the bone graft granules. It is believed that this prevents excessive dehydration during the subsequent mixing step. Following this, the more concentrated second aqueous calcium phosphate suspension is mixed in. The resultant composition exhibits excellent clinical handling properties and cohesiveness. These improvements in cohesiveness allow the use of synthetic bone substitute graft granules, whilst addressing the previous issue of synthetic granules falling down the narrow neo-medullary canal. Moreover, as the calcium phosphate paste promotes cell proliferation of the bone formation cells, its presence in the composition as whole helps to promote ingrowth of blood vessels and invasion by bone cells, leading to the replacement of the composition by new viable bone.

Conveniently, the bone graft granules have an average diameter of larger than 1 mm. Preferably, the bone graft granules have an average diameter in the range of 2-4 mm. This provides the best granule size for packing the medullary canal.

Conveniently, the bone graft granules are a synthetic bone substitute. Due to the greatly enhanced cohesiveness provided by the calcium phosphate paste binder, the composition can use predominantly or entirely synthetic bone substitute materials. This thereby avoids the problems of disease transmission and high cost associated with allograft bone materials, without compromising clinical handling.

Preferably, the bone graft granules comprise hydroxyapatite (HAP). Hydroxyapatite has a high hardness and toughness, making it particularly suitable for impaction grafting techniques, where tight packing is desired. The bone graft granules may also comprise tricalcium phosphate (TCP). The bone graft granules may also comprise autograft, allograft, or xenograft bone.

In one embodiment the bone graft granules comprise demineralised bone matrix (DBM).

Preferably, the first aqueous calcium phosphate suspension is mixed with the bone graft granules to give a composition of ratio 30-50:50-70 first aqueous calcium phosphate suspension to bone graft granules by weight. In a preferred embodiment the first aqueous calcium phosphate suspension is mixed with the bone graft granules to give a composition of ratio 30:50 first aqueous calcium phosphate suspension to bone graft granules by weight. In an alternative embodiment, the first aqueous calcium phosphate suspension is mixed with the bone graft granules to give a composition of ratio 40:60 first aqueous calcium phosphate suspension to bone graft granules by weight. It has been found that these quantities allow the first aqueous calcium phosphate suspension to particularly effectively coat the bone graft granules during the first mixing step, resulting in a final composition having improved handling properties.

Preferably, the second aqueous calcium phosphate suspension is mixed with the intermediate mixture at a composition of ratio 20-40:60-80 second aqueous calcium phosphate suspension to bone graft granules by weight. More preferably, the second aqueous calcium phosphate suspension is mixed with the intermediate mixture at a composition of ratio 30:70 second aqueous calcium phosphate suspension to bone graft granules by weight. It has been found that these quantities result in a final composition having particularly improved handling properties and cohesiveness.

Conveniently, calcium phosphate is present at a concentration of 5 wt % to 20 wt % in said first aqueous calcium phosphate suspension. Preferably, calcium phosphate is present at a concentration of 12 wt % to 18 wt % in said first aqueous calcium phosphate suspension. More preferably, calcium phosphate is present at a concentration of 13 wt % to 17 wt % in said first aqueous calcium phosphate suspension. It has been found that these concentrations are particularly effective at coating the bone graft granules during the first mixing step, resulting in a final composition having improved handling properties.

Conveniently, calcium phosphate is present at a concentration of 20 wt % to 40 wt % in said second aqueous calcium phosphate suspension. Preferably, calcium phosphate is present at a concentration of 20 wt % to 30 wt % in said second aqueous calcium phosphate suspension. In one embodiment, calcium phosphate is present at a concentration of 26 wt % in said second aqueous calcium phosphate suspension. It has been found that these concentrations result in a final composition having particularly improved handling properties and cohesiveness.

Conveniently, said first and second aqueous calcium phosphate suspensions comprise calcium phosphate nano-particles. Due to the large surface area of these particles, osteogenesis is enhanced.

Conveniently, said calcium phosphate nano-particles are crystalline.

Preferably, the crystalline calcium phosphate nano-particles are fully crystalline.

Optionally, the composition may further comprise growth factors and/or therapeutic agents. In this way, the resultant composition can be provided with additional components, depending on its application, to further improve clinical results.

Conveniently, the composition comprises more than 35 wt % water.

According to a further aspect of the preset invention there is provided a pre-filled container comprising the above composition. In this way, a complete, ready to use, product is provided in a pre-filled container, such as a pre-filled syringe or jar, which can be easily used by a surgeon to apply the bone repair composition.

According to a further aspect of the preset invention there is provided a method for producing a bone repair composition comprising steps of: mixing a first aqueous calcium phosphate suspension with bone graft granules to form a intermediate mixture; and mixing the intermediate mixture with a second aqueous calcium phosphate suspension; wherein said first aqueous calcium phosphate suspension contains a lower weight concentration of calcium phosphate than the second aqueous calcium phosphate suspension.

Conveniently, the bone graft granules have an average diameter of larger than 1 mm. Preferably, the bone graft granules have an average diameter in the range of 2-4 mm.

Conveniently, the bone graft granules are a synthetic bone substitute. Preferably, the bone graft granules comprise hydroxyapatite (HAP). The bone graft granules may also comprise tricalcium phosphate (TCP). The bone graft granules may also comprise autograft, allograft, or xenograft bone.

Preferably, the first aqueous calcium phosphate suspension is mixed with the bone graft granules to give a composition of ratio 30-50:50-70 first aqueous calcium phosphate suspension to bone graft granules by weight. In a preferred embodiment the first aqueous calcium phosphate suspension is mixed with the bone graft granules to give a composition of ratio 30:50 first aqueous calcium phosphate suspension to bone graft granules by weight. In an alternative embodiment, first aqueous calcium phosphate suspension is mixed with the bone graft granules to give a composition of ratio 40:60 first aqueous calcium phosphate suspension to bone graft granules by weight.

Conveniently, the second aqueous calcium phosphate suspension is mixed with the intermediate mixture at a composition of ratio 20-40:60-80 second aqueous calcium phosphate suspension to bone graft granules by weight. Preferably, the second aqueous calcium phosphate suspension is mixed with the intermediate mixture at a composition of ratio 30:70 second aqueous calcium phosphate suspension to bone graft granules by weight.

Conveniently, calcium phosphate is present at a concentration of 5 wt % to 20 wt % in said first aqueous calcium phosphate suspension. Preferably, calcium phosphate is present at a concentration of 12 wt % to 18 wt % in said first aqueous calcium phosphate suspension. Most preferably, calcium phosphate is present at a concentration of 13 wt % to 17 wt % in said first aqueous calcium phosphate suspension. In one embodiment, calcium phosphate is present at a concentration of 14 wt % in said first aqueous calcium phosphate suspension.

Conveniently, calcium phosphate is present at a concentration of 20 wt % to 40 wt % in said second aqueous calcium phosphate suspension. Preferably, calcium phosphate is present at a concentration of 20 wt % to 30 wt % in said second aqueous calcium phosphate suspension. In one embodiment, calcium phosphate is present at a concentration of 26 wt % in said second aqueous calcium phosphate suspension.

Conveniently, said first and second aqueous calcium phosphate suspensions comprise calcium phosphate nano-particles.

Conveniently, said calcium phosphate nano-particles are crystalline.

Preferably, the crystalline calcium phosphate nano-particles are fully crystalline.

Optionally, the method may further comprise the step of mixing in growth factors and/or therapeutic agents.

Conveniently, the resultant composition comprises more than 35 wt % water.

According to a further aspect of the present invention, there is provided a composition for forming a neo-medullary canal in revision hip surgery, said composition formed by firstly mixing a first aqueous calcium phosphate suspension with bone graft granules to form an intermediate mixture, and secondly mixing the intermediate mixture with a second aqueous calcium phosphate suspension, wherein said first aqueous calcium phosphate suspension contains a lower weight concentration of calcium phosphate than the second aqueous calcium phosphate suspension.

Illustrative examples of the present invention will now be described below in detail.

In this connection, a method of preparing a bone grafting composition according to an embodiment of the present invention will now be described.

Firstly, an aqueous stock solution (suspension) of ˜8% w/w of calcium phosphate nano particles is heated to dry it. As it dries, the relative concentration of calcium phosphate increases until two calcium phosphate pastes are formed, the first paste having a concentration of 13-17% w/w and the second having a concentration of 20-30% w/w. In this specific embodiment, the pastes have a concentration of approximately 14% w/w for the first paste, and 26% w/w for the second paste. The concentration is measured by weighing an oven-dried sample of the mixture until a constant weight is reached. In an alternative embodiment, rather than heat drying, “vacuum filtration” could be used to obtain the desired paste concentrations.

In this connection, the aqueous suspension of calcium phosphate nano particles contains fully crystalline calcium phosphate phases, such as hydroxyapatite, tri-calcium phosphate, or tri-calcium orthophosphate. This crystalline structure means that the calcium phosphate does not self-harden in the presence of water and, hence, the suspension remains as a paste or putty, rather than forming a hardened solid. In this embodiment, the aqueous stock suspension is hydroxyapatite nano-paste. As such, the pure hydroxyapatite has a hexagonal crystal structure and an acicular habit of nanometer sized crystals forming clusters, i.e. needle shaped crystals. The chemical formula for this is Ca₁₀(PO₄)₆(OH)₂ and the Ca:P ratio is 1.67.

As bone graft granules, hydroxyapatite granules of 2-4 mm particle size are weighed and thoroughly mixed with an amount of the first calcium phosphate paste (˜14 wt %) to give a composition of ratio 40:60 first calcium phosphate paste to hydroxyapatite by weight.

Once the above intermediate mixture is fully mixed, an amount of second calcium phosphate paste (˜26 wt %) is then added to the intermediate mixture to obtain a ratio of 30:70 second calcium phosphate paste to hydroxyapatite by weight. This is then thoroughly mixed to produce the final composition.

Mechanical testing of the cohesion of the final composition will now be described. The composition was placed in a cylinder mould with an internal diameter of 17 mm diameter and a height of 40 mm. A 1 kg weight, comparable to an operative hammer, was dropped 20 times from a height of 50 mm on a piston to compact the composition. The mould was split lengthwise to carefully remove the impacted sample. The height of all the samples was measured after impaction.

The cylindrical samples were transferred to a 5 KN servo-hydraulic testing machine (manufactured by ESH Testing Ltd, Brierley Hill, UK). The specimens were loaded at a strain rate of 2.5% of the initial sample height per minute, to a maximum of 15% of sample height or until failure was achieved. Stress-strain diagrams were then compiled from the results and from these, the compressive strength at failure or at 15% strain was determined. The sample size, loading rate and definition of failure were chosen according to an international standard. The cohesion or shear strength at zero total normal stress for each sample was then calculated as half the compressive strength. The above procedure was repeated three times to give an average cohesion value. All statistical analyses were performed using Systat 11 (Systat Software Inc., Richmond, Calif.). The cohesion values from three experiments were 20, 20 and 25, giving a mean value of 21.7 kPa.

The above recorded cohesion values for the present invention are comparative to comparative samples formed of allograft and clotted blood. In contrast, however, comparative samples of allograft without clotted blood, allograft and synthetic mixtures, and synthetics achieve much lower cohesion values, as shown in Table 1 below.

TABLE 1
Mean cohesion values for each separate
experimental group. From Oakley J & Kuiper JH. JBJS
Vol 88B (No 6) June 2006, 828-831.

| Bone (%) | Extender type | Clotted blood added | Cohesion (KPa) |
|---|---|---|---|
| 100 | — | No | 11.9 |
| 100 | — | Yes | 23.7 |
| 50 | HAP | No | 0.9 |
| 50 | HAP | Yes | 0.5 |
| 50 | HAP/TCP | No | 3.5 |
| 50 | HAP/TCP | Yes | 10.6 |
| 0 | HAP | No | 0 |
| 0 | HAP/TCP | Yes | 0 |

Accordingly, with the present invention, the calcium phosphate paste enables particulate bone grafts, such as synthetic bone substitute granules, to be used in impaction bone grafting where cohesion between the particles is required.

It has been particularly found that the two step mixing process employed in the present invention achieves pronounced improvements in cohesion between the bone graft granules. In contrast, comparative examples using a single step mixing procedure exhibited much lower cohesion. It is believed this is because the first mixing step, using a lower calcium phosphate paste concentration, provides a thin coating over the granules which prevents excessive dehydration when the second paste is added.

Furthermore, as discussed above, with the present invention, the nano particles of calcium phosphate are crystalline. As such, the composition remains paste-like and fluid once mixed. This allows the calcium phosphate nano particles and the bone graft granules disbursed therein to remain mobile within the resultant composition, permitting movement thereof as well as bone ingrowth. This avoids limiting the expression of the components' osteoinductive function. As a result, the composition can achieve high levels of osteoinduction.

Furthermore, in the application of impaction grafting, a further important property of the composition, along with cohesion, is the ability for bone cement to penetrate into the bone repair composition. This can be measured in mm and affects the stability of an implant after implantation. That is, before bone ingrowth occurs, the bone cement used to stabilise the joint between the implant and the newly impacted bone grafts. If bone cement is unable to penetrate into the bone repair composition, effective bonding between the implant and the bone will not occur. Conversely, if the penetration of the bone cement is too high, the bone repair composition may be unable to work effectively to promote bone ingrowth. Accordingly, for impaction grafting, it is preferable that the bone repair composition has a penetration values of between approximately 1 mm-2 mm, along with cohesion value of 5-25 KPa.

As an illustration, the following results were achieved with specific examples of the present invention.

TABLE 2
Cohesion and Penetration values for the application of impaction grafting.

| First Aqueous Calcium Phosphate | | Second Aqueous Calcium | | | |
|---|---|---|---|---|---|
| Suspension | | Phosphate Suspension | | | |
| | Mix Ratio with | | Mix Ratio with | | |
| Concentration | Hydroxyapatite | Concentration | Hydroxyapatite | Cohesion | Penetration |
| (% w/w) | granules | (% w/w) | granules | (KPa) | (mm) |
| 13% | 30:70 | 30% | 30:70 | 10 | 2 |
| 13% | 50:50 | 25% | 30:70 | 10 | 1.5 |
| 15% | 30:70 | 25% | 30:70 | 8 | 2 |
| 15% | 30:70 | 30% | 30:70 | 20 | 1 |
| 15% | 50:50 | 20% | 30:70 | 12 | 1.25 |
| 17% | 50:50 | 25% | 30:70 | 10 | 1.5 |

Although the present invention has been described in the above illustrated embodiment, the present invention is not limited solely to this particular embodiment.

For example, in the above embodiment, hydroxyapatite has been used as the bone graft granules, although it will be understood that other materials could also be used, or mixtures of granules could be used. For example, materials such as tricalcium phosphate granules, other synthetic bone substitutes, or harvested bone such as autograft, allograft, or xenograft bone.

Demineralised bone matrix (hereinafter DBM) could also be used as the bone graft granules. In this connection, DBM is typically provided in the form of a fine powder, with particle sizes of 74-420 μm. However, as a consequence of this, DBM can be extremely difficult to handle in clinical applications. The present invention allows the calcum phosphate to be used as a carrier to enhance cohesion between the DBM particles and thereby provide better handling properties of the DBM.

In this connection, for example, a first aqueous crystalline calcium phosphate suspension of 14% w/w is firstly mixed with DBM to form an intermediate mixture. After this, a second aqueous crystalline calcium phosphate suspension of 25% w/w is mixed into the intermediate mixture. Preferably, the first and second aqueous calcium phosphate components are mixed with the DBM to give a composition ratio of 40-60:20-40:10-30, by weight, first aqueous crystalline calcium phosphate component to second aqueous crystalline calcium phosphate component to the DBM, respectively. In a particularly preferred embodiment the first and second aqueous crystalline calcium phosphate components are mixed with the DBM to give a composition ratio of 50:30:20, by weight, first aqueous crystalline calcium phosphate component to second aqueous crystalline calcium phosphate component to the DBM, respectively.

Furthermore, different bone graft granule sizes could be used to optimise handling properties and characteristics depending on surgeon preference or the particular clinical application. Similarly, the concentrations and quantities of the first and second calcium phosphate suspensions can be varied to alter the properties of the final composition.

Moreover, it will be understood that further mixing steps could be introduced, for example to introduce additional agents such as growth factors and therapeutic agents.

Finally, although the above example describes the manufacture of a bone treatment composition for impaction bone grafting in joint replacement surgery, it will also be understood that the present invention could be used for other bone repair applications, for example to repair bone cysts or bone voids.

1-53. (canceled)

54. A method for producing a bone repair composition comprising steps of:
mixing a first aqueous calcium phosphate suspension with bone graft granules to form a intermediate mixture; and
mixing the intermediate mixture with a second aqueous calcium phosphate suspension,
wherein said first and second aqueous calcium phosphate suspensions comprise crystalline calcium phosphate nano-particles, and
wherein said first aqueous calcium phosphate suspension contains a lower weight concentration of calcium phosphate than the second aqueous calcium phosphate suspension.

55. The method of claim 54 wherein the bone graft granules comprise at least one selected from the group of: demineralised bone matrix (DBM), a synthetic bone substitute, hydroxyapatite (HAP), tricalcium phosphate (TCP), autograft, allograft, and xenograft bone.

56. The method of claim 54 wherein the first aqueous calcium phosphate suspension is mixed with the bone graft granules to give a composition of ratio 30-50:50-70 first aqueous calcium phosphate suspension to bone graft granules by weight.

57. The method of claim 54 wherein the first aqueous calcium phosphate suspension is mixed with the bone graft granules to give a composition of ratio 30:50 first aqueous calcium phosphate suspension to bone graft granules by weight.

58. The method of claim 54 wherein the first aqueous calcium phosphate suspension is mixed with the bone graft granules to give a composition of ratio 40:60 first aqueous calcium phosphate suspension to bone graft granules by weight.

59. The method of claim 54 wherein the second aqueous calcium phosphate suspension is mixed with the intermediate mixture at a composition of ratio 20-40:60-80 second aqueous calcium phosphate suspension to bone graft granules by weight.

60. The method of claim 54 wherein the second aqueous calcium phosphate suspension is mixed with the intermediate mixture at a composition of ratio 30:70 second aqueous calcium phosphate suspension to bone graft granules by weight.

61. The method of claim 54 wherein calcium phosphate is present at a concentration of 5 wt % to 20 wt % in said first aqueous calcium phosphate suspension.

62. The method of claim 54 wherein calcium phosphate is present at a concentration of 12 wt % to 18 wt % in said first aqueous calcium phosphate suspension.

63. The method of claim 54 wherein calcium phosphate is present at a concentration of 20 wt % to 40 wt % in said second aqueous calcium phosphate suspension.

64. The method of claim 54 wherein calcium phosphate is present at a concentration of 20 wt % to 30 wt % in said second aqueous calcium phosphate suspension.

65. The method of claim 54, wherein the crystalline calcium phosphate nano-particles are fully crystalline.

66. The method of claim 54 further comprising the step of mixing in growth factors and/or therapeutic agents.

67. The method of claim 54 wherein the resultant composition comprises more than 35 wt % water.

68. A bone repair composition formed by firstly mixing a first aqueous calcium phosphate suspension with bone graft granules to form an intermediate mixture, and secondly mixing the intermediate mixture with a second aqueous calcium phosphate suspension,
wherein said first aqueous calcium phosphate suspension contains a lower weight concentration of calcium phosphate than the second aqueous calcium phosphate suspension.

69. The composition of claim 68 wherein the bone graft granules comprise at least one selected from the group of: demineralised bone matrix (DBM), a synthetic bone substitute, hydroxyapatite (HAP), tricalcium phosphate (TCP), autograft, allograft, and xenograft bone.

70. The composition of claim 68 further comprising growth factors and/or therapeutic agents.

71. The composition of claim 68 wherein the composition comprises more than 35 wt % water.

72. The composition of claim 68 wherein the crystalline calcium phosphate nano-particles are fully crystalline.

73. A composition for forming a neo-medullary canal in revision hip surgery, said composition formed by firstly mixing a first aqueous calcium phosphate suspension with bone graft granules to form an intermediate mixture, and secondly mixing the intermediate mixture with a second aqueous calcium phosphate suspension,
wherein said first and second aqueous calcium phosphate suspensions comprise crystalline calcium phosphate nano-particles, and
wherein said first aqueous calcium phosphate suspension contains a lower weight concentration of calcium phosphate than the second aqueous calcium phosphate suspension.
